SEQUENCE LISTING (4 sequences)

SEQ ID NO: 1 - protein, 30 aa, Artificial Sequence
  chemically synthesized
NGMIGEVVYQRAVMAVGSLTIKRIVTEKTD

SEQ ID NO: 2 - protein, 19 aa, Artificial Sequence
  chemically synthesized
DLLEKIAEDRVEFNEDGDR

SEQ ID NO: 3 - protein, 23 aa, Artificial Sequence
  chemically synthesized
WSLRRDPRGAPAWARGSRPRHAS

SEQ ID NO: 4 - protein, 22 aa, Artificial Sequence
  chemically synthesized
TINEERSEIVWSLRRDPRGAPA

(19) United States
(12) Patent Application Publication
(10) Pub. No.: US 2010/0210816 A1
(43) Pub. Date: Aug. 19, 2010

(54) NMDAR Biomarkers for Diagnosing and Treating Cerebral Ischemia

(76) Inventor: Svetlana Dambinova, Atlanta, GA (US)

Correspondence Address: PATENT CORRESPONDENCE;ARNALL GOLDEN GREGORY LLP, 171 17TH STREET NW, SUITE 2100, ATLANTA, GA, 30363, US

(21) Appl. No.: 12/518,629

(22) PCT Filed: Dec. 12, 2007
(86) PCT No.: PCT/US07/87278

§ 371 Date: Jun. 10, 2009

Related U.S. Application Data
(60) Provisional application No. 60/874,356, filed on Dec. 12, 2006.

(51) Int. Cl. C07K 14/00; C07K 7/00; C07K 16/00; C07H 21/02; C07H 21/04

(52) U.S. Cl. 530/324; 530/326; 530/391.3; 536/23.1

(57) ABSTRACT

Provided are methods for detecting various subunits and isoforms of NMDA receptors to help diagnose and differentiate (1) the anatomical location of NMDA receptor over-expression, and (2) ischemic conditions in the central and peripheral nervous systems. Further provides are therapeutic strategies and interventions for the treatment and prevention of stroke, cardiovascular disease, and ischemic lesions in the brain and peripheral nervous system, and pulmonary disorders based on the results of such diagnoses.

### RELATIONSHIP TO PRIOR APPLICATIONS

This application claims priority to U.S. Provisional Application No. 60/874,356, filed Dec. 12, 2006.

### FIELD OF THE INVENTION

This invention relates to methods of diagnosing and treating brain injury due to cerebral or central ischemia.

### BACKGROUND OF THE INVENTION

The N-methyl-d-aspartate subtype of glutamate receptor (NMDAR or NMDA receptor) serves critical functions in physiological and pathological processes in the central and peripheral nervous system, including neuronal development, plasticity and neurodegeneration. Various investigators have reported that the receptor consists of three primary subunits: NR1, NR2A-D, and NR3A-B, and that the coexpression of NR3A with NR1 and NR2 subunits modulates NMDAR activity in pre- and postsynaptical membranes.

The postsynaptic NR2 subfamily consists of four individual subunits, NR2A to NR2D. In situ hybridization has revealed overlapping but different expression for NR2 mRNA. In particular, NR2A mRNA is distributed ubiquitously like NR1 with highest densities occurring in hippocampal regions. In contrast, NR2B is expressed predominantly in the forebrain but not in the cerebellum where NR2C predominates (Parsons et al., Drug News Perspect. 1998 November; 11(9):523-569). The spinal cord reportedly expresses high levels of NR2C and NR2D (Tolle et al., J Neurosci. 1993 December; 13(12):5009-28) and these may form heteroligomeric receptors with NR1 plus NR2A (Sundstrom et al., Exp Neurol. 1997 December; 148(2):407-13).

NR3 is reportedly expressed predominantly in the developing central nervous system and does not seem to form functional homomeric glutamate-activated channels (Sucher et al., J Neurosci. 1995 October; 15(10):6509-20). From in situ and immunocytochemical analyses, it is known that NR3B is expressed predominantly in motor neurons, whereas NR3A is more widely distributed.

Zukin et al. have reported that alternative splicing generates eight isoforms for the pre- and postsynaptic NR1 subfamily (Zukin and Bennett, Trends Neurosci. 1995 July; 18(7):306-13). The variants arise from splicing at three exons; one encodes a 21-amino acid insert in the N-terminal domain (N1, exon 5), and two encode adjacent sequences of 37 and 38 amino acids in the C-terminal domain (C1, exon 21 and C2, exon 22). NRI variants are sometimes denoted by the presence or absence of these three alternatively spliced exons (from N to C1 to C2). NR1₁₁₁ has all three exons, NR1₀₀₀ has none, and NR1₁₀₀ has only the N-terminal exon. The variants from NR1₀₀₀ to NR1₁₁₁ are alternatively denoted as NMDAR1e, c, d, a, g, f, h and b respectively or NMDAR1-4a,-2a,-3a,-1a,-4b,-2b,-3b and-1b respectively, but the more frequent terminology uses non-capitalized subscripts, which suffices for the most common splice variants, i.e. NR1a (NR1₀₁₁ or NMDAR1A) and NR1b (NR1₁₀₀ or NMDARIG). NR1a receptors are more concentrated in rostral structures such as the cortex, caudate, and hippocampus, while NR1b receptors are principally found in more caudal regions such as the thalamus, colliculi, locus coeruleus and cerebellum (Laurie et al., Brain Res Mol Brain Res. 1995 August; 32(1):94-108).

The role of NMDA receptors has been explored by numerous investigators. For example, it has been reported that the process of peripheral and central sensitization is maintained, at least theoretically and experimentally, through the excitatory neurotransmitter glutamate, which is believed to be released when the NMDA receptor is activated (Gudin, Medscape Neurology & Neurosurgery 2004). In addition, available evidence suggests that the roles of NMDA receptors differ with respect to the processing of visceral and somatic pain. One set of authors have concluded that NMDA receptors are present in peripheral visceral nerves and may be important in visceral pain processing in the absence of inflammation, thus providing a novel mechanism for development of peripheral sensitization and visceral hyperalgesia (McRoberts et al., Gastroenterology 2001;120:1737-1748).

In a number of studies, blocking NMDA receptors has been proposed as a preventive treatment for protecting neurons from ischemia (Dugan L L and Kim-Han J S In:Basic Neurochemistry. Siegel et al. Eds, 2006, 7^{th} edition, 559-73). However, blocking NMDA receptors may be detrimental to animals and humans (Davis et al, Stroke 2000; 31:347-354; Ikonomidou et al, Proc. Natl. Acad. Sci. U.S.A. 2000; 97:12885-12890). Moreover, although blocking excitotoxicity of NMDA receptors has proven effective in laboratory models of disease, clinical trials of neurorrotective therapies have generally failed to benefit patients (Lees et al. (2000) Lancet 355:1949-1954). These failures are generally attributed to side effects of glutamate receptor antagonists which may evoke failure of high brain functions (mental disturbances, memory decline and asocial behavior).

Some limited efforts have been made at using natural peptides derived from the brain for treating cerebral ischemic events (Gusev, Skvortsova. Brain Ischemia. NY-Boston-Dordecht-London: Kluwer Academic/Plenum Publishers, 2003; 382). For example, it has been shown in clinical trials that ACTG hormone 4-10 fragment (Semax) drastically improves movement and mental performance in patients who have suffered an acute stroke. Cerebrolyzin, an extract of small peptides from pig brain, has shown positive clinical effect optimizing energetic metabolism of nervous cells and Ca²⁺ homeostasis. It has also been shown that cerebrolysin in a dose of 10 mg daily reduces lipid peroxidation and the accumulation of glutamate receptor antibodies, thereby improving patient memory, speech and psychological activity (www.consilium-medicum.com).

Recently, NMDAR peptides and their antibodies have been proposed for the treatment of stroke and epilepsy (During et al, Science, 2000, 287:1453-60) and as biomarkers of neurotoxicity underlying cerebral ischemia and stroke (Dambinova S A, et al. Stroke 2002; 33:1181-1182; Dambinova S A, et al. Clin Chem 2003; 49:1752-1762). With neuronal death or ischemia, NR2 peptide fragments of the NMDA receptor break off and appear in the bloodstream and generate an antibody response. Dambinova et al. have reported that the peptide fragments and antibodies can both be detected in blood samples (Dambinova S A, et al. Stroke 2002). They have further reported that adult patients who have suffered an acute ischemic stroke have elevated blood levels of NR2 peptide/Ab that correlate with the amount of brain damage revealed through brain scans (MRI) and neurocognitive testing (Dambinova S A, et al. Clin Chem 2003; 49:1752-1762).

This work defines a new approach for diagnosing and treating cerebral ischemic events in which excitotoxicity is a subset of a larger framework of brain cell it jury that involves NMDA receptor peptides and antibodies.

### OBJECTS OF THE INVENTION

It is an object of the present invention to identify the anatomical location of NMDAR over-expression in a human based on the subunit or isoform of NMDAR that is detected.

It is another object of the invention to provide methods and biochemical markers for distinguishing between metabolic imbalances in the pulmonary system, the peripheral nervous system, and the brain, based on the over-expression of NMDA receptor subunits and subsequent trafficking of NMDA receptor subunits in the bloodstream.

It is another object of the invention to provide methods and biochemical markers for diagnosing ischemic conditions in humans, and for distinguishing between schemic conditions in the brain and ischemic conditions in the peripheral nervous system.

### SUMMARY OF THE INVENTION

The present invention provides strategies for diagnosing and treating ischemic microvascular events in the central nervous system. These strategies target NMDA receptors involved in the neurotoxic cascade caused by ischemic microvascular events, and are based on the discovery of novel NMDAR isoforms released to the bloodstream from oxygen deprived tissues, and the location in the human body where particular NMDAR isoforms are expressed. NMDAR isoforms have been revealed by HPLC (on the basis of different retention time), electrofocusing (various protein pI) and immunoblot of peptides using monoclonal antibodies to phosphorylated or acetylated isoform peptide fragments. Glutamate receptor isoforms targeted by this invention include recombinant and/or mutant subtypes that are optionally modified through acetylation or phosphorylation.

In particular, the inventors have discovered that particular recombinant and/or mutant NR1, NR2 and NR3 are overexpressed in different anatomical regions of the human body (Table 1), and that when these subunits are overexpressed the proteolysis activated production of peptide fragments that enter the bloodstream are recognized as foreign antigens by the immune system, which responds by generating detectable amounts of autoantibodies. The existence and location of NMDAR over-expression in the human body can thus be ascertained by detecting NMDAR isoform fragments or antibodies in bodily fluids such as the bloodstream, and by determining the particular subunit or isoform of NMDAR that is present.

The inventors have also discovered therapeutic strategies which, when combined with the diagnostic methods of the present invention, can be practiced more effectively. In particular, the inventors have discovered that the markers of the present invention can be used to select drug therapies, and to monitor the effectiveness and progress of drug therapies for the prevention and treatment of certain conditions described herein.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION OF THE DRAWINGS

The accompanying drawings, which are incorporated in and constitute a part of this specification, illustrate several embodiments of the invention and together with the description, serve to explain the principles of the invention.

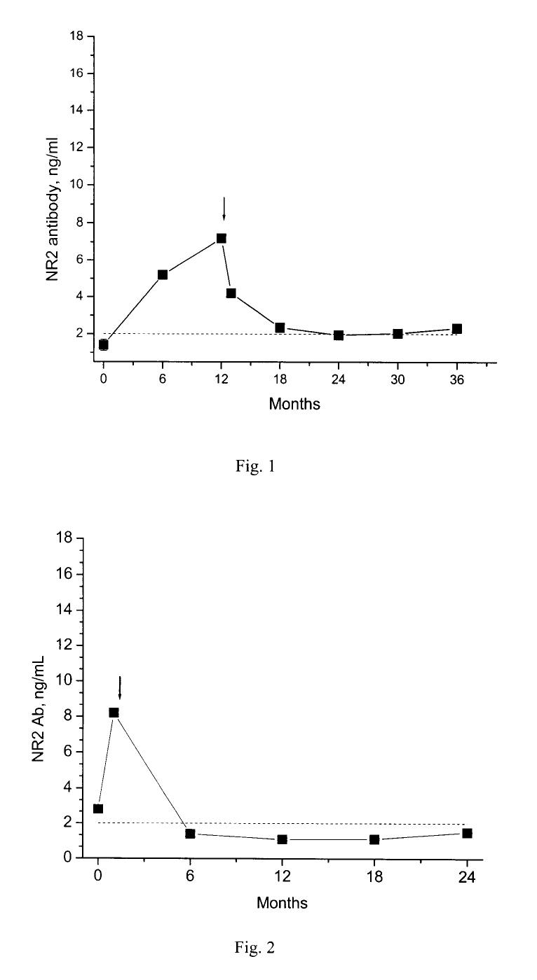

FIGS. 1 through 3 plot the NR2 Ab levels in individual patients before and after therapeutic intervention as described more particularly in examples 1 and 2.

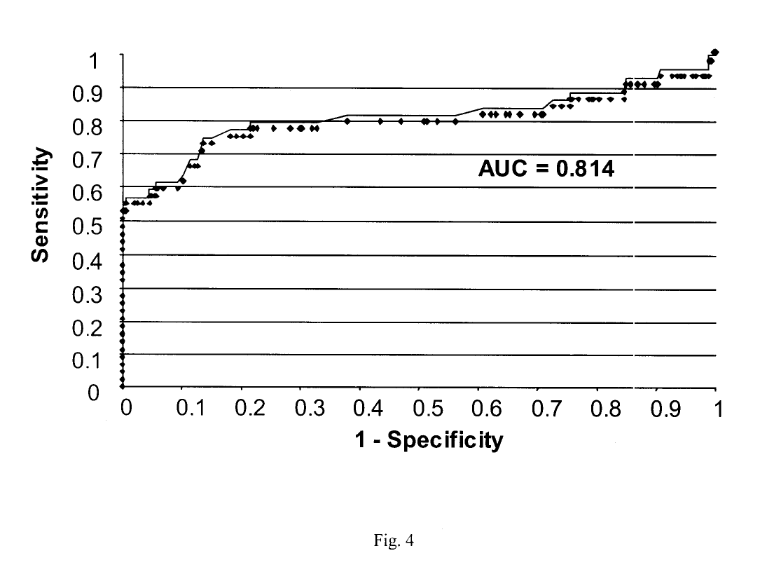

FIG. 4 is an ROC curve plotting diagnostic sensitivity versus diagnostic specificity, based on a comparison of pre-operative NR2 Ab levels and post-operative neurological adverse.

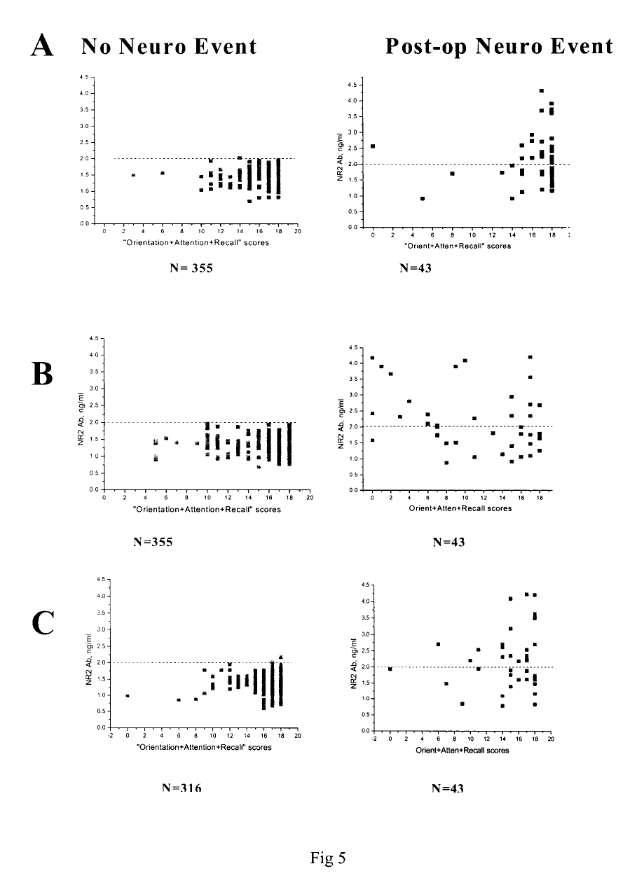

FIG. 5 is a graphical depiction of the distribution of NR2 Ab in serum samples of patients without neurological adverse events and those with neurological complications detected pre-op (A), at 24 hours (B), and 48 hours (C) after surgery. Data presented in pre-operative values of NR2 Ab vs. combined scores of MMSE components: orientation+attention+recall. The dotted line shows a cut off of 2.0 ng/ml.

### DETAILED DESCRIPTION OF THE INVENTION

The present invention may be understood more readily by reference to the following detailed description of preferred embodiments of the invention and the Examples included therein.

### Definitions and Use of Terms

As used in this specification and in the claims which follow, the singular forms “a,” “an” and “the” include plural referents unless the context clearly dictates otherwise. Thus, for example, reference to “a fragment” includes mixtures of fragments, reference to “an cDNA oligonucleotide” includes more than one oligonucleotide, and the like.

“Polypeptide,” “protein” and “peptide” are used interchangeably herein and include a molecular chain of amino acids linked through peptide bonds. The terms do not refer to a specific length of the product. Thus, “peptides,” “oligopeptides,” and “proteins” are included within the definition of polypeptide. The terms include post-translational modifications (isoforms) of the polypeptide, for example, glycosylations, acetylations, phosphorylations, chelates, and the like. In addition, protein fragments, analogs, mutated or variant proteins, chimeric peptides and the like are included within the meaning of polypeptide. The polypeptide, protein and peptides may be in cyclic form or they may be in linear form. In one particular embodiment, the peptides of the current invention are deglycosylated, or dephosphorylated from their natural state, or are prepared synthetically without naturally occurring glycosylation or phosphorylation.

An NMDA receptor or NMDAR is one of a family of ligand-gated ion channels that bind preferentially to N-methyl-D-aspartate and that mediate the vast majority of excitatory neurotransmission in the brain (Dingledine R. et al., Pharmacol Rev. 1999 March; 51(1):7-61.). The receptors include subunits reported in the literature as NR1, NR2A, NR2B, NR2C, NR2D, NR3A and NR3B, that perform distinct pharmacological functions. GenEMBL Accession Nos. have been reported for NR1 (X58633), NR2A (U09002) and NR2B (U28861), and are described in WO 02/12892 to Dambinova.

An NMDA receptor peptide refers to a full length NMDA receptor protein, a peptide fragment of the naturally or synthetically occurring full length NMDA receptor, or an anologue or isoform thereof. An NR2 peptide thus includes the full length NR2A, NR2B, NR2C and NR2D subunits, in addition to fragments, analogs and derivatives thereof. Similarly, an NR2A, NR2B, NR2C, or NR2D peptide means the full length naturally occurring NR2A, NR2B, NR2C or NR2D peptide subunits, or a fragment, analog or derivative thereof. The N-terminal domain of NMDA peptides refers to the amino acid N-terminal domain fragment of the full length peptide, or a fragment analog or derivative thereof, typically about 40 or 50 amino acids long, but as much as 150, 200 or 300 amino acids long, as described in WO 02/12892 to Dambinova.

As used herein, the terms “antagonist” and “natural peptide containing z nc, Fe³⁺ and or magnesium,” when used in the context of modulating a binding interaction (such as the binding of a glutamate, glycine and polyamine domain sequences to the N-terminal fragment of natural or synthetic NMDA receptor sequence), are used interchangeably and refer to an agent that reduces the binding of the, e.g., N-terminal fragment of natural or synthetic NMDA receptor sequence and the, e.g., domain peptide.

An “analogue” of a peptide means a peptide that contains one or more amino acid substitutions, deletions, additions, or rearrangements. For example, it is well known in the art of protein biochemistry that an amino acid belonging to a grouping of amino acids having a particular size or characteristic (such as charge, hydrophobicity, and hydrophilicity) can often be substituted for another amino acid without altering the activity of the protein, particularly in regions of the protein that are not directly associated with biological activity. Thus, an analogue of an NMDA peptide is useful in the present invention if it includes amino acid substitutions, deletions, additions or rearrangements at sites such that antibodies raised against the analogue are still specific against the NMDAR peptide.

Unless stated to the contrary, an NMDAR analogue or mutant as used in this document refers to a sequence that has at least 80% amino acid identity with naturally occurring NMDA, although it could also contain at least 85%, 90%, or 95% identity. Amino acid identity is defined by an analogue comparison between the analogue or mutant and naturally occurring NMDA. The two amino acid sequences are aligned in such a way that maximizes the number of amino acids in common along the length of their sequences; gaps in either or both sequences are permitted in making the alignment in order to maximize the number of common amino acids. The percentage amino acid identity is the higher of the following two numbers: (1) the number of amino acids that the two peptides have in common with the alignment, divided by the number of amino acids in the NMDA analogue, multiplied by 100, or (2) the number of amino acids that the two peptides have in common with the alignment, divided by the number of amino acids in naturally occurring NMDA peptide, multiplied by 100. Amino acids appended to the ends of the fragment under analysis are not taken into consideration.

NMDA derivatives include naturally occurring NMDA and NMDA analogues and fragments thereof that are chemically or enzymatically derivatized at one or more constituent amino acids, including side chain modifications, backbone modifications, and N- and C-terminal modifications, by for example acetylation, hydroxylation, methylation, amidation, phosphorylation or glycosylation. The term also includes NMDA salts such as zinc NMDA and ammonium NMDA.

A protein or peptide is measured “directly” in the sense that the protein or peptide is itself measured in the biological sample, as opposed to some other indirect measure of the protein or peptide such as autoantibodies to the protein or peptide, or cDNA or mRNA associated with the expression of the protein or peptide.

The term “antibody” is synonymous with “immunoglobulin.” As used herein, the term “antibody” includes both the native antibody, monoclonally generated antibodies, polyclonally generated antibodies, recombinant DNA antibodies, and biologically active derivatives of antibodies, such as, for example, Fab′, F(ab′)₂ or Fv as well as single-domains and single-chain antibodies. A biologically active derivative of an antibody is included within this definition as long as it retains the ability to bind the specified antigen. Thus, an NR2 antibody that binds specifically to an NR2 peptide has the ability to bind at least one NR2 peptide. In one particular embodiment, the immunoglobulins of the current invention are deglycosylated or dephosphorylated from their natural state, or are prepared synthetically without naturally occurring glycosylation or phosphorylation.

When ranges are given by specifying the lower end of a range separately from the upper end of the range, it will be understood that the range can be defined by selectively combining any one of the lower end variables with any one of the upper end variables that is mathematically possible.

### Discussion

In a first principal embodiment the invention provides methods and kits for determining the anatomical source of NMDAR isoform expression in the human body, and the pathological process leading to such over-expression. The location of such over-expression, the pathological process leading to such over-expression, the diseased state associated with such over-expression, and the particular NMDAR isoform that is overexpressed, are all described in greater detail in Table 1. The markers can be used to distinguish between any of the tissues, processes or disease states identified in Table 1.

TABLE 1
Source of Recombinant and/or Mutant NMDAR

| NMDAR | | | |
|---|---|---|---|
| Isoform | Tissue | Process | Disease |
| NR2A/NR2B | Brain | Ischemic lesion | TIA/ischemic |
| | | | stroke |
| NR1/NR3 | Brain, | Hypoxia, | Lacunar stroke |
| | pulmonary | microemboli | |
| | system | | |
| NR3/NR2C | Pulmonary | Lung lesions | Asthma, |
| NR3/NR2D | system | | pneumonia, |
| | | | tuberculosis |
| NR1/GluR1 | Heart | Atrial Fibrilation | TIA/Ischemic |
| | | | Stroke |

The terminology employed in this document is designed to describe recombinant isoforms. Thus, for example, an NR1/NR2 peptide refers to a peptide that combines sequences from the NRI NMDA receptor subtype and the NR2 NMDA receptor subtype. The sequences are preferably autoantigenic, and preferably derive from the N-terminal domain of the recited NMDA receptor subtype. The peptides are preferably less than about 100, 60 or 40 amino acids in length, and greater than about 10, 15 or 20 amino acids. It will of course be understood that analogs of such sequences may also be present in the recombinant peptide.

One of the most useful aspects of the invention is the ability to diagnose the anatomical source of ischemia when presented with a patient suffering from an apparent ischemic stroke or transient ischemic attack. Thus, for example, over-expression of the NR1/NR3 peptide suggests a lacunar stroke, and that the stroke potentially originates from microemboli or hypoxia in the pulmonary system or the brain. Therefore, in one embodiment the invention provides a method of diagnosing a patient suspected of suffering a stroke or cerebral ischemia comprising: (a) directly or indirectly testing a biological fluid from said patient for an amount of NR1/NR3 recombinant peptide or an analog thereof; (b) optionally comparing said amount of NR1/NR3 recombinant NMDAR peptide fragments with a designated standard for said recombinant NMDAR peptide; and (c) optionally correlating an excess amount of said NR1/NR3 recombinant peptide or analog thereof with one or more diagnoses selected from lacunar stroke, NMDAR over-expression in the brain or pulmonary system, hypoxia or microemboli in the brain, or hypoxia or microemboli in the pulmonary system.

Said designated standard preferably refers to a population norm in apparently healthy human subjects, or a previously recorded value of NR1/NR3 for said patient. Alternatively, said designated standard may simply refer to a non-detectable quantity of peptide or analog thereof. Population norms for NR1/NR3 peptides range generally from 0.01 to 1.0 ng/ml of plasma and a cutoff may be selected from any figure between these two endpoints including 0.01, 0.025, 0.05, 0.075, 0.1, 0.25, 0.5, 0.75 or 1.0 ng/ml of plasma. Population norms for antibodies specific for NR1/NR3 peptides generally range from 0.1 to 10.0 ng/ml of plasma, and a cutoff may be selected from any figure between these two endpoints including 0.1, 0.25, 0.5, 0.75, 1.0, 2.5, 5.0, 7.5 or 10.0 ng/ml of plasma.

In another embodiment the methods of the invention employ NR3/NR2C or NR3/NR2D recombinant peptides, and over-expression of the peptides is used to diagnose a patient suffering from pulmonary dysfunction such as asthma, pneumonia or tuberculosis. In this embodiment the invention provides a method of diagnosing a patient suspected of suffering a stroke or cerebral ischemia comprising: (a) directly or indirectly testing a biological fluid from said patient for an amount of NR3/NR2C or NR3/NR2D recombinant peptide or an analog thereof; (b) optionally comparing said amount of NR3/NR2C or NR3/NR2D recombinant NMDAR peptide fragments to a designated standard for said NMDAR peptide; and (c) optionally correlating an excess amount of said NR3/NR2C or NR3/NR2D recombinant peptide or analog thereof with NMDAR over-expression in the pulmonary system or lesions in the lung.

Said designated standard preferably refers to a population norm in apparently healthy human subjects, or a previously recorded value of NR3/NR2C or NR3/NR2D for said patient. Alternatively, said designated standard may simply refer to a non-detectable quantity of peptide or analog thereof. Population norms for NR3/NR2C and NR3/NR2D peptides range generally range from 0.01 to 1.0 ng/ml of plasma and a cutoff may be selected from any figure between these two endpoints including 0.01, 0.025, 0.0.5, 0.075, 0.1, 0.25, 0.5, 0.75 or 1.0 ng/ml of plasma. Population norms for antibodies specific for NR1/NR3 peptides generally range from 0.1 to 10.0 ng/ml of plasma, and a cutoff may be selected from any figure between these two endpoints including 0.1, 0.25, 0.5, 0.75, 1.0, 2.5. 5.0, 7.5 or 10.0 ng/ml of plasma.

In yet another embodiment the methods of the invention employ an NR1/GluR1 recombinant NMDAR peptide, and the over-expression of the peptide is used to diagnose stroke or cerebral ischemia resulting from cardiac dysfunction such as atrial fibrillation. In this embodiment the invention provides a method of diagnosing a patient suspected of suffering a stroke or cerebral ischemia comprising: (a) directly or indirectly testing a biological fluid from said patient for an amount of NR1/GluR1 recombinant peptide or an analog thereof; and (b) optionally comparing said amount of NR1/GluR1 recombinant NMDAR peptide fragments with a designated standard; and (c) optionally correlating an excess amount of said NR1/GluR1 recombinant peptide or analog thereof with NMDAR over-expression in the heart, or atrial fibrillation in the heart.

Said designated standard preferably refers to a population norm in apparently healthy human subjects, or a previously recorded value of NR1/GluR1 for said patient. Alternatively, said designated standard may simply refer to a non-detectable quantity of peptide or analog thereof. Population norms for NR1/GluR1 peptides range generally from 0.01 to 1.0 ng/ml of plasma and a cutoff may be selected from any figure between these two endpoints including 0.01, 0.025, 0.05, 0.075, 0.1, 0.25, 0.5, 0.75 or 1.0 ng/ml of plasma. Population norms for antibodies specific for NR1/GluR1 peptides generally range from 0.1 to 10.0 ng/ml of plasma, and a cutoff may be selected from any figure between these two endpoints including 0.1, 0.25, 0.5, 0.75, 1.0, 2.5, 5.0, 7.5 or 10.0 ng/ml of plasma.

Still other embodiments pertain to the use of these receptors and their biological markers in diagnostic kits, and to the use of the markers in diagnosing various pathological conditions and anatomical locations associated with over-expression of NMDA receptors. Such methods are preferably carried out by measuring two or more of the recombinant or mutant peptides at the same time, and are preferably carried out through the use of a kit that allows one to test for two or more of the recombinant or mutant peptides. Therefore, in another embodiment the invention provides a method of diagnosing NMDAR over-expression in a human subject comprising:
a) detecting in a bodily fluid, directly or indirectly, the amount of two or more recombinant NMDAR peptide fragments selected from:
   i) an NR2A/NR2B recombinant peptide or analog thereof;
   ii) an NR1/NR3 recombinant peptide or analog thereof;
   iii) an NR3/NR2C recombinant peptide or analog thereof;
   iv) an NR3/NR2D recombinant peptide or analog thereof; and
   v) an NR1/GluR1 recombinant peptide or analog thereof,
b) optionally comparing said amount of two or more recombinant NMDAR peptide fragments with a designated standard for said recombinant NMDAR peptide fragments; and
c) optionally correlating an excess amount of one or more recombinant NMDAR peptide fragments with an anatomical location of NMDAR over-expression in the patient.

Said designated standard for NR2A/NR2B preferably refers to a population norm in apparently healthy human subjects, or a previously recorded value of NR2A/NR2B for said patient. Alternatively, said designated standard may simply refer to a non-detectable quantity of peptide or analog thereof. Population norms for NR2A/NR2B peptides range generally from 0.01 to 1.0 ng/ml of plasma and a cutoff may be selected from any figure between these two endpoints including 0.01, 0.025, 0.05, 0.075, 0.1, 0.25, 0.5, 0.75 or 1.0 ng/ml of plasma. Population norms for antibodies specific for NR2A/NR2B peptides generally range from 0.1 to 10.0 ng/ml of plasma, and a cutoff may be selected from any figure between these two endpoints including 0.1, 0.25, 0.5, 0.75, 1.0, 2.5, 5.0, 7.5 or 10.0 ng/ml of plasma.

In one particular embodiment the invention provides a method of distinguishing between ischemic conditions in the central and peripheral nervous systems by measuring a recombinant NR1/NR3 peptide or analog thereof or a recombinant NR1/GluR1 peptide or analog thereof; and correlating an excessive level of said recombinant peptide to ischemia in the peripheral nervous system. Once again, excessive levels can be determined by comparison with a designated standard such as a population norm in apparently healthy human subjects.

In another embodiment the invention provides a kit comprising two or more recombinant NMDAR peptide fragments, or two or more antibodies that specifically bind to recombinant NMDAR peptide fragments, or two or more nucleic acids that encode recombinant NMDAR peptide fragments, each bound to a diagnostic substrate or indicator reagent, wherein said two or more recombinant NMDAR peptide fragments are selected from:
a) an NR2A/NR2B recombinant peptide or analog thereof;
b) an NR1/NR3 recombinant peptide or analog thereof;
c) an NR3/NR2C recombinant peptide or analog thereof;
d) an NR3/NR2D recombinant peptide or analog thereof; and
e) an NR1/GluR1 recombinant peptide or analog thereof

In still another embodiment, the invention provides an isolated recombinant or mutant NMDAR peptide, or an isolated antibody specific for said recombinant or mutant NMDAR peptide, wherein said recombinant or mutant peptide is selected from:

a) an NR1/NR3 recombinant peptide or analog thereof;

b) an NR3/NR2C recombinant peptide or analog thereof; and

c) an NR3/NR2D recombinant peptide or analog thereof.

In another embodiment the invention provides an isolated antibody that is specific for the above-mentioned recombinant peptides (a)-(c), or a nucleic acid that encodes the recombinant peptides. In still another embodiment the peptide, antibody or nucleic acid is bound to a diagnostic substrate or an indicator reagent.

The term “isolated” excludes instances wherein the peptide or antibody may have been separated from other peptide bands, as in gel electrophoresis, but the peptide or antibody has not been physically isolated from the gel or the other peptide bands. The peptide can, of course, be part of a much larger peptide, as contemplated by the “comprising” terminology. In a preferred embodiment, however, the peptide is exactly as represented, or an analog thereof, optionally bound through an appropriate linker to a diagnostic substrate (such as a plate, a particle or a bead) or to an indicator reagent. Similarly, the antibodies and nucleic acids of the present invention are preferably specific for the exact sequences represented (or analogs thereof), and are optionally bound through an appropriate linker to a diagnostic substrate or an indicator reagent.

Preferred amino acid sequences for the recombinant NMDAR peptides discussed above are set forth below in Table 2.

TABLE 2
NMDA Receptor Isoforms

| NMDAR Isoform | Amino Acid Sequences |
|---|---|
| NR2A/NR2B | NGMIGEVVYQRAVMAVGSLTIKRIVTEKTD 31 |
| | (SEQ ID 4) |
| NR1/NR3 | DLLEKIAEDRVEFNEDGDR 19 |
| | (SEQ ID 5) |
| NR3/NR2C | WSLRRDPRGAPAWARGSRPRHAS 23 |
| | (SEQ ID 6) |
| NR3/NR2D | TINEERSEIVWSLRRDPRGAPA 22 |
| | (SEQ ID 7) |

Therefore, in a first principal embodiment, the invention provides an isolated peptide comprising SEQ ID 4, SEQ ID 5, SEQ ID 6 or SEQ ID 7, or an analog thereof. In a second principal embodiment the invention provides an isolated antibody that is specific for, or a nucleic acid that encodes, SEQ ID 4, SEQ ID 5, SEQ ID 6 or SEQ ID 7, or an analog thereof (i.e. of such sequence). In still another embodiment the peptide, antibody or nucleic acid is bound to a diagnostic substrate or an indicator reagent.

The methods of the present invention can be performed using practically any biological fluid where circulating cerebral NMDA receptors, or markers of such receptors, are expressed or found, including blood, urine, blood plasma, blood serum, cerebrospinal fluid, saliva, perspiration or brain tissue. In a preferred embodiment the biological fluid is plasma or serum, and in an even more preferred embodiment the plasma or serum is diluted to a ratio of about 1:50.

In still another embodiment the invention provides methods for treating or preventing the NMDAR over-expression described above. These methods further include the administration of one or more agents designed to improve the flow and absorption of oxygen by the affected tissue, and subsequent measurements of the relevant NMDAR peptide to evaluate the effectiveness of the therapy (typically one or more times every three, six or twelve months). Drugs that are useful for practicing these methods include lipid lowering agents, blood viscocity reducing agents, antiplatelet agents, anticoagulants, blood pressure reduction agents, and tissue plasminogen activator (tPA).

Exemplary antiplatelet agents for practicing the methods of the present invention include aspirin, abciximab, cilostazol, clopidogrel bisulfate, dipyridamole, eptifibatide, ticlodipine, and tirofiban. Exemplary anticoagulants include dalteparin, danaparoid, enoxaparin, heparin, tinzaparin, and warfarin. Exemplary blood pressure medications include diuretics such as amiloride, bumetanide, chlorothiazide, chlorthalidone, furosemide, hydrochlorothiazide, indapamide, and spironolactone; angiotensin-converting enzyme (ACE) inhibitors such as benazepril, captopril, enalapril, fosinopril, lisinopril, moexipril, perindopril, quinapril, ramipril, and trandolapril; and angiotensin-2 receptor antagonists such as candesartan, eprosartan, irbesartan, losartan, telmisartan, and valsartan. Exemplary lipid lowering agents include clofibrate, gemfibrozil, nicotinic acid, the statins (including atorvastatin, fluvastatin, lovastatin, pravastatin, rosuvastatin, and simvastatin), and nicotinic acid.

Pentoxifylline (marketed commercially as Trental®), or cilostazol (marketed commercially as Pletal®), by increasing red blood cell pliability and reducing blood viscosity, can be used with success as well. Useful doses of pentoxifilline, when administered orally, fall within the following ranges on a daily basis (including or excluding one or both endpoints): 400-1600; 800-1200; 400-500; 450-550; 500-600; 550-650; 600-700; 650-750; 700-800; 750-850; 800-900; 850-950; 900-100; 950-1050; 1000-1100; 1050-1150; 1100-1200; 1150-1250; 1200-1300; 1250-1350; 1300-1400; 1350-1450; 1400-1500; and 1450-1550. Useful doses of cilostazol, when administered orally, fall within the following ranges on a daily basis (including or excluding one or both endpoints): 50-300 mg; 50-90 mg; 100-200 mg; 100-150 mg; 150-200 mg, and 200-250 mg.

While these therapies are useful in the treatment of any of the conditions described in Table A, they are particularly useful in the treatment or prevention of ischemic stroke or transient ischemic attack, and for recovering from a TIA or ischemic stroke. These latter methods rely primarily upon the evaluation of NR2 levels because of this peptide's specificity for ischemic conditions in the brain.

The therapy can be initiated for any of the following three types of patients:
apparently healthy subjects, who have never suffered a confirmed TIA or ischemic stroke, based on NR2 testing that indicates that a person is at heightened risk for suffering a stroke or TIA;
patients who are confirmed to be suffering an ischemic stroke or TIA based on the results of NR2 testing, in whom the development of an ischemic lesion is ongoing. Patients in this category will typically be tested for NMDAR no more than about three hours, six hours or twelve hours after the onset of stroke or TIA symptoms; and
patients who have suffered an ischemic stroke or TIA who are attempting to recover from the stroke or TIA, or to prevent another stoke or TIA. Patients attempting to recover from a stroke or TIA may initiate treatment at any time, but preferably will initiate treatment within 24 hours, 48 hours, 72 hours, 1 week, or 2 weeks of the incidence of the stroke or TIA.

In this embodiment the invention provides a method of treating, preventing or recovering from a stroke or transient ischemic attack in a human subject comprising: (a) directly or indirectly testing a bodily fluid from said subject for a pretreatment amount of a NR2A/NR2B recombinant peptide or analog thereof; (b) comparing said pretreatment amount with a population norm of a direct or indirect measure of said recombinant peptide derived from apparently healthy human subjects; and (c) if a level of said recombinant peptide in excess of said population norm is detected, administering a drug selected from lipid lowering agents, blood viscocity reducing agents, antiplatelet agents, anticoagulants, blood pressure reduction agents, and tissue plasminogen activator (tPA).

In each of these scenarios, a decision to administer therapy using the methods of the present invention is based on a predetermined cutoff of peptide levels. Thus, for example, it has been experimentally and clinically shown that levels of recombinant and/or mutant NR2 (NR2A/NR2B or NR1/NR2) circulating peptide in plasma or serum that are greater than 200, 100, or 50 pg/ml, or levels of NR2 antibodies of greater than 2.0, 1.8, 1.5, or 1.0 ng/ml, indicate that the patient would benefit from the treatment methods of the present invention. In contrast, levels of recombinant and/or mutant NR2 peptide in plasma or serum that are less than 200, 100, or 50 pg/ml, or levels of NR2 circulating antibodies of less than 2.0, 1.8, 1.5, or 1.0 ng/ml, indicate that the patient would not benefit from the treatment methods of the present invention. Antibody levels generally range from 0.5 to 3.0 ng/ml or 1.0 to 2.0 ng/ml. Peptide levels generally range from 0.5 to 3.0 ng/ml or 1.0 to 2.0 ng/ml. A preferred peptide cut off is 100 pg/ml and antibody cutoff is 2.0 ng/ml.

In each of these scenarios, the patient preferably undergoes additional testing to monitor and evaluate the effectiveness of the treatment regimen, and to modify the treatment as necessary. Therefore, in another embodiment, the invention further provides: (d) testing a bodily fluid from said subject for a subsequent amount of NR2 peptides or antibodies one or more additional times; (e) comparing said subsequent amounts with the amount of NR2 peptides or antibodies detected in step (b). If reductions in NR2 peptides or antibodies are seen, the patient can continue therapy as prescribed.

Of course, the frequency of additional monitoring will vary depending on the type of patient being treated and the risk that the patient will suffer an ischemic event or stroke. For example, a patient suffering a stroke would likely be tested a second time very shortly after administration of said therapy (i.e. within 30, 15 or even 5 minutes of administration). tPA is a particularly preferred medication for administering to patients suffering from an ongoing stroke (i.e. within about 6, 4 or 2 hours of stroke onset).

For patients at risk for an ischemic event or stroke, or who are attempting to recover from an ischemic stroke or TIA, the patient would likely be tested shortly after treatment was initiated to judge the effectiveness of the treatment, and to modify the treatment until NR2 peptide/antibodies levels reached control levels of cut off. Patients would typically be monitored a second time within about 1 month or 3 months of the initial monitoring event, and subsequently at intervals not to exceed every 6 or 12 months.

### Treatment Platform

The compounds of the invention may be administered to a subject per se or in the form of a sterile composition or a pharmaceutical composition. Pharmaceutical compositions comprising the compounds of the invention may be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries that facilitate processing of the active peptides or peptide analogues into preparations which can be used pharmaceutically Proper formulation is dependent upon the route of administration chosen.

For topical administration the compounds of the invention can be formulated as solutions, gels, ointments, creams, suspensions, etc. as are well-known in the art. Systemic formulations include those designed for administration by injection, e.g. subcutaneous, intravenous, intramuscular, intranasal, intrathecal or intraperitoneal injection, as well as those designed for transdermal, transmucosal, oral or pulmonary administration.

For injection, the compounds of the invention can be formulated in aqueous solutions, preferably in physiologically compatible buffers such as Hanks's solution, Ringer's solution, or physiological saline buffer. The solution can contain formulatory agents such as suspending, stabilizing and/or dispersing agents. Alternatively, the compounds can be in powder form for constitution with a suitable vehicle, e.g., sterile pyrogen-free water, before use.

For oral administration or vaccination, the compounds can be readily formulated by combining the active peptides (antibodies) or peptide analogues with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. For oral solid formulations such as, for example, powders, capsules and tablets, suitable excipients include fillers such as sugars, such as lactose, sucrose, mannitol and sorbitol; cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP); granulating agents; and binding agents. If desired, disintegrating agents may be added, such as the cross-linked polyvinylpyrrolidone, agar, or alginic acid or a salt thereof such as sodium alginate. If desired, solid dosage forms may be sugar-coated or enteric-coated using standard techniques.

For oral liquid preparations such as, for example, suspensions, elixirs and solutions, suitable carriers, excipients or diluents include water, glycols, oils, alcohols, etc. Additionally, flavoring agents, preservatives, coloring agents and the like may be added. For buccal administration, the compounds may take the form of tablets, lozenges, etc. formulated in conventional manner.

For administration by inhalation, the compounds for use according to the present invention are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Alternatively, other pharmaceutical delivery systems may be employed. Liposomes and emulsions are well known examples of delivery vehicles that may be used to deliver peptides and peptide analogues of the invention. Certain organic solvents such as dimethylsulfoxide also may be employed, although usually at the cost of greater toxicity. Additionally, the compounds may be delivered using a sustained-release system, such as semipermeable matrices of solid polymers containing the therapeutic agent. Various of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

As the compounds of the invention may contain charged side chains or termini, they may be included in any of the above-described formulations as the free bases or as pharmaceutically acceptable salts. Pharmaceutically acceptable salts are those salts which substantially retain the biologic activity of the free bases and which are prepared by reaction with inorganic acids. Pharmaceutical salts tend to be more soluble in aqueous and other protic solvents than are the corresponding free base forms.

The compounds of the invention will generally be used in an amount effective to achieve the intended purpose (e.g., treatment of central or peripheral neuronal injury). The therapies of the invention are performed by administering the subject drug in a therapeutically effective amount. By therapeutically effective amount is meant an amount effective ameliorate or prevent the symptoms, or prolong the survival of, the patient being treated. Determination of a therapeutically effective amount is well within the capabilities of those skilled in the art, especially in light of the detailed disclosure provided herein. An “therapeutic amount” or “therapeutic concentration” of a NMDAR isoforms or antibodies is an amount that reduces binding activity to receptor by at least about 40%, preferably at least about 50%, often at least about 70%, and even as much as at least about 90%. Binding can be measured in vitro (e.g., in an assay) or in situ.

For systemic administration, a therapeutically effective dose can be estimated initially from in vitro assays. For example, a dose can be formulated in animal models to achieve a circulating concentration range that includes the IC50. Such information can be used to more accurately determine useful doses in humans.

Initial dosages can also be estimated from in vivo data, e.g., animal models, using techniques that are well known in the art. One having ordinary skill in the art could readily optimize administration to humans based on animal data. Dosage amount and interval may be adjusted individually to provide plasma levels of the compounds that are sufficient to maintain therapeutic effect. Usual patient dosages for administration by injection range from about 0.1 to 10 mg/day, preferably from about 0.5 to 1 mg/day. Therapeutically effective serum levels may be achieved by administering multiple doses each day. For usual peptide/antibodies therapeutic treatment of cerebral ischemic events within 6 hours of event is typical.

In cases of local administration or selective uptake, the effective local concentration of the compounds may not be related to plasma concentration and should be optimized therapeutically effective local dosages without undue experimentation. The amount of compound administered will, of course, be dependent on the subject being treated, on the subject's weight, the severity of the affliction, the manner of administration and the judgment of the prescribing physician. The therapy may be repeated intermittently while symptoms detectable or even when they are not detectable. The therapy may be provided alone or in combination with other drugs.

Preferably, a therapeutically effective dose of the compounds described herein will provide therapeutic benefit without causing substantial toxicity. Toxicity of the compounds described herein can be determined by standard pharmaceutical procedures in experimental animals, e.g., by determining the LD50 (the dose lethal to 50% of the population) or the LD100 (the dose lethal to 100% of the population). The dose ratio between toxic and therapeutic effect is the therapeutic index. Compounds which exhibit high therapeutic indices are preferred. The data obtained from these cell culture assays and animal studies can be used in formulating a dosage range that is not toxic for use in human. The dosage of the compounds described herein lies preferably within a range of circulating concentrations that include the effective dose with little or no toxicity. The dosage may vary within this range depending upon the dosage form employed and the route of administration utilized. The exact formulation, route of administration and dosage can be chosen by the individual physician in view of the patient's condition. (See, e.g., Fingl et al., 1975, In: The Pharmacological Basis of Therapeutics, Ch. 1, p. 1).

### Diagnostic Platforms

The diagnostic methods of the present invention can be performed using any number of known diagnostic techniques, including direct or indirect ELISA, RIA, immunodot, immunoblot, latex aggutination, lateral flow, fluorescence polarization, and microarray. In one particular embodiment, the invention is practiced using an immobilized solid phase for capturing and measuring the NMDAR peptide marker. Therefore, in one embodiment the methods of the invention comprise: (a) contacting a biological sample from the patient with an immobilized solid phase comprising a NMDAR peptide or antibody, for a time sufficient to form a complex between said NMDAR peptide or antibody and NMDAR antibody or peptide in said biological sample; (c) contacting said complex with an indicator reagent attached to a signal-generating compound to generate a signal; and (d) measuring the signal generated. In a preferred embodiment, the indicator reagent comprises chicken anti-human or anti-human IgG attached to horseradish peroxidase.

In a preferred embodiment, the solid phase is a polymer matrix. More preferably, the polymer matrix is polyacrylate, polystyrene, or polypropylene. In one preferred embodiment the solid phase is a microplate. In another preferred embodiment, the solid phase is a nitrocellulose membrane or a charged nylon membrane.

In another embodiment, the method is performed using agglutination. Therefore, in still another embodiment the invention comprises: (a) contacting a biological sample from the patient with an agglutinating carrier comprising a NMDAR peptide or antibody, for a time sufficient to form an agglutination complex between said NMDAR peptide or antibody and NMDAR antibody or peptide in said biological sample; (c) generating a signal from the agglutination; (d) correlating said signal to said levels of one or more markers of NMDAR peptide or antibody. In a preferred embodiment, the “sufficient time” is less than 30, 20, 15 or even 10 minutes.

Latex agglutination assays have been described in Beltz, G. A. et al., in Molecular Probes: Techniques and Medical Applications, A. Albertini et al., eds., Raven Press, New York, 1989, incorporated herein by reference. In the latex agglutination assay, antibody raised against a particular biomarker is immobilized on latex particles. A drop of the latex particles is added to an appropriate dilution of the serum to be tested and mixed by gentle rocking of the card. With samples lacking sufficient levels of the biomarkers, the latex particles remain in suspension and retain a smooth, milky appearance. However, if biomarkers reactive with the antibody are present, the latex particles clump into visibly detectable aggregates.

An agglutination assay can also be used to detect biomarkers wherein the corresponding antibody is immobilized on a suitable particle other than latex beads, for example, on gelatin, red blood cells, nylon, liposomes, gold particles, etc. The presence of antibodies in the assay causes agglutination, similar to that of a precipitation reaction, which can then be detected by such techniques as nephelometry, turbidity, infrared spectrometry, visual inspection, colorimetry, and the like.

The term latex agglutination is employed generically herein to refer to any method based upon the formation of detectable agglutination, and is not limited to the use of latex as the immunosorbent substrate. While preferred substrates for the agglutination are latex based, such as polystyrene and polypropylene, particularly polystyrene, other well-known substrates include beads formed from glass, paper, dextran, and nylon. The immobilized antibodies may be covalently, ionically, or physically bound to the solid-phase immunoadsorbent, by techniques such as covalent bonding via an amide or ester linkage, ionic attraction, or by adsorption. Those skilled in the art will know many other suitable carriers for binding antibodies, or will be able to ascertain such, using routine experimentation.

Conventional methods can be used to prepare antibodies for use in the present invention. For example, by using a peptide of a NMDA protein, polyclonal antisera or monoclonal antibodies can be made using standard methods. A mammal, (e.g., a mouse, hamster, or rabbit) can be immunized with an immunogenic form of the peptide which elicits an antibody response in the mammal. Techniques for conferring immunogenicity on a peptide include conjugation to carriers or other techniques well known in the art. For example, the peptide can be administered in the presence of adjuvant. The progress of immunization can be monitored by detection of antibody titers in plasma or serum. Standard ELISA or other immunoassay procedures can be used with the immunogen as antigen to assess the levels of antibodies. Following immunization, antisera can be administered and, if desired, polyclonal antibodies isolated from the sera.

To produce monoclonal antibodies, antibody producing cells (lymphocytes) can be harvested from an immunized animal and fused with myeloma cells by standard somatic cell fusion procedures thus immortalizing these cells and yielding hybridoma cells. Such techniques are well known in the art, (e.g., the hybridoma technique originally developed by Kohler and Milstein (Nature 256, 495-497 (1975)) as well as other techniques such as the human B-cell hybridoma technique (Kozbor et al., Immunol. Today 4, 72 (1983)), the EBV-hybridoma technique to produce human monoclonal antibodies (Cole et al. Monoclonal Antibodies in Cancer Therapy (1985) Allen R. Bliss, Inc., pages 77-96), and screening of combinatorial antibody libraries (Huse et al., Science 246, 1275 (1989)). Hybridoma cells can be screened immunochemically for production of antibodies specifically reactive with the peptide and the monoclonal antibodies can be isolated. Therefore, the invention also contemplates hybridoma cells secreting monoclonal antibodies with specificity for NMDAR proteins or fragments as described herein.

In one embodiment the method is practiced using a kit that has been calibrated at the factory based upon antibodies purified from human blood. Therefore, in another embodiment the invention is practiced under the following conditions: (a) NMDAR antibody levels in said biological fluid are measured using a diagnostic kit; (b) said diagnostic kit comprises bound NMDAR peptides; and (c) said kit is manufactured against an antibody standard comprising a fraction of immunoglobulins G purified from human blood.

In addition, the method can be practiced using commercially available chemiluminescence techniques. For example, the method could employ a two-site sandwich immunoassay using direct chemiluminescent technology, using constant amounts of two monoclonal antibodies. The first antibody, in a fluid reagent, could be an acridinium ester labeled monoclonal mouse anti-human NMDA receptor peptide BNP (F(ab′)₂ fragment specific to a first portion of the peptide. The second antibody, in the solid phase, could be a biotinylated monoclonal mouse anti-human antibody specific to another portion of the peptide, which could be coupled to streptavidin magnetic particles. An immuno-complex would be formed by mixing a patient sample and the two antibodies. After any unbound antibody conjugates are washed away, the chemiluminescence of the immuno-complex signal could then be measured using a luminometer.

When the NMDA receptors are detected indirectly, by measuring the cDNA expression of the NMDA receptors, the measuring step in the present invention may be carried out by traditional PCR assays such as cDNA hybridization, Northern blots, or Southern blots. These methods can be carried out using oligonucleotides encoding the polypeptide antigens of the invention. Thus, in one embodiment the methods of this invention include measuring an increase of NMDAR cDNA expression by contacting the total DNA isolated from a biological sample with oligonucleotide primers attached to a solid phase, for a sufficient time period. In another preferred embodiment, NMDAR cDNA expression is measured by contacting an array of total DNA bound to a solid matrix with a ready-to-use reagent mixture containing oligonucleotide primers for a sufficient time period. Expressed NMDAR cDNA is revealed by the complexation of the cDNA with an indicator reagent that comprises a counterpart oligonucleotide to the cDNA attached to a signal-generating compound. The signal-generating compound is preferably selected from the group consisting of horseradish peroxidase, alkaline phosphatase, urinase and non-enzyme reagents. The signal-generating compound is most preferably a non-enzyme reagent.

The immunosorbent of the present invention for measuring levels of autoantibody can be produced as follows. A fragment of the receptor protein is fixed, preferably by covalent bond or an ionic bond, on a suitable carrier such as polystyrene or nitrocellulose. If the standard polystyrene plate for immunological examinations is employed, it is first subjected to the nitration procedure, whereby free nitrogroups are formed on the plate surface, which are reduced to amino groups and activated with glutaric dialdehyde serving as a linker. Next the thus-activated plate is incubated with about 2 to 50 nM of the target peptide for the purpose of chemically fixing the respective immunogenic fragment of the receptor protein for a time and at a temperature sufficient to assure fixation (i.e. for about 16 hours at 4° C.).

It is also practicable to produce the immunosorbent by fixing the respective fragment of the receptor protein on nitrocellulose strips by virtue of ionic interaction. The respective fragment of the receptor protein isolated from the mammals' brain is applied to nitrocellulose and incubated for 15 min at 37° C. Then nitrocellulose is washed with a 0.5% solution of Tween-20, and the resultant immunosobent is dried at room temperature and stored in dry place for one year period.

### Examples

The following examples are put forth so as to provide those of ordinary skill in the art with a complete disclosure and description of how the compounds claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at room temperature, and pressure is at or near atmospheric.

### Example 1

### Combined Use of NMDAR Test and Antiplatelet Therapy

The FDA recently approved the use of aspirin to prevent stroke in men and women who have already had a stroke or mini-stroke to decrease the likelihood of a second stroke. A large meta-analysis showed that aspirin reduces the incidence of stroke, myocardial infarction, or vascular death by 25%. (Collaborative overview of randomized trials of antiplatelet therapy I: Prevention of death, myocardial infarction and stroke by prolonged antiplatelet therapy in various categories of patients. Antiplatelet Trialists Collaboration. BMJ 1994; 308:81-106.)

Aspirin also is an established treatment for the prevention of cerebrovascular accidents (CVA) in patients with transitory ischemic attacks (TIA) or minor cerebral vascular accident (CVA). It reduces the risk of TIA by 20%. (Kase C S. Antiaggregant treatment: present and future Rev Neurol. 1999; 28:1013-6 Article in Spanish). However some patients still have a vascular event even if they are taking aspirin. Researchers have found that some patients are predisposed to biochemical aspirin resistance. (Gum P A, Marchant K K, Welsh P A, et al. A prospective, blinded determination of the natural history of aspirin resistance among stable patients with cardiovascular disease. J Am Coll Cardiol 2003; 41:961-965.)

In this study, serial measurements of blood levels of NR2 antibodies in an out-patient setting in individuals with diabetes mellitus (DM) and atherosclerosis (AS) who are at high risk for subsequent CVA were evaluated to establish whether this biomarker can be used to monitor the efficacy of aspirin treatment. Every person who participated in the study underwent neurological examination and computed tomography (CT) of the brain. The age of the 33 persons (17 male/16 female) ranged from 47-64 yrs (mean 55.7 yrs). Patients with documented 5-10 year history of DM/AS and normal neurological examination without a previous history of neurological event such as transient ischemic attack (TIA), or prolonged reversible ischemic neurological deficit or stroke were observed. Based on laboratory and clinical testing, all participants were at borderline glucose intolerance, with blood glucose levels of 119 to 132 mg/dL, and had complaints on fatigue, low concentration of attention, and numbness in digits of left or right hand.

Out of 22 individuals, 10 (6 male/4 female) were randomly assigned to a placebo group and the rest used daily over-counter aspirin in a dose of 325 mg which formed group 1 (Table 3). None of people of group 1 developed the resistance to aspirin. Group 2 individuals with DM/AS (n=9) who were resistant to aspirin (325 mg/day) were revealed from a retrospective study of 186 persons at high risk for subsequent CVA performed at Clinic of Pavlov' Medical University (St. Petersburg, Russia) in 2001-2002.

TABLE 3
Cumulative detection of NR2 antibodies over 24 months of aspirin treatment follow up

| | | NR2 antibodies (ng/ml) over 24 months of follow up | | | | | TIA |
|---|---|---|---|---|---|---|---|
| Group | N | 0 mo. | 6 mo. | 12 mo. | 18 mo. | 24 mo. | rate |
| Placebo | 10 | 2.91 ± 0.35 | 2.84 ± 0.12 | 2.77 ± 0.19 | 2.95 ± 0.19 | 3.21 ± 0.45 | 1/10 |
| Group 1 | 12 | 3.05 ± 0.21 | 2.51 ± 0.28 | 2.33* ± 0.32 | 1.92* ± 0.25 | 1.60** ± 0.25 | 0 |
| Group 2 | 9 | 2.87 ± 0.11 | 3.22 ± 0.30 | 3.51* ± 2.2 | 4.05* ± 0.21 | 5.14** ± 0.33 | 2/9 |

*P < 0.01 and **P < 0.001 compared with basic level of placebo and corresponding group.

NR2 antibodies were detected at a base level for each group before treatment was initiated (0 mo.) and then each following 6 months for two years. All three groups of individuals demonstrated close initial levels of NR2 antibodies that exceeded the cut off of 2 ng/ml by about 43.5-52.5%%. Follow up after treatment of the placebo group showed insignificant alterations in antibodies contents during the 24 months of treatment. A TIA event was documented in 1 patient from the placebo group. The levels of NR2 antibodies were significantly reduced (below cut off of 2 ng/ml) in patients in group 1 with no CVA consequences in 1 year of treatment follow up. Patients from group 2 resistant to aspirin had no improvement in NR2 antibodies levels at any time during the study. By the end of the study at 24 mo., two out of 9 persons suffered a TIA which developed into an acute ischemic stroke in one patient.

### Example 2

### Combined Use of NMDAR Test and Pentoxifylline Therapy

Trental (pentoxifylline) is an antiplatelet drug that improves oxygen supplies to tissues by stimulating vasodilation and inhibiting platelet aggregation. It reduces blood viscosity and the risk of a new blood clot forming in the brain.

There is some evidence for the effectiveness of the Trental in the routine management of acute ischemic stroke (non-significant reduction of odds of early death—odds ratio 0.64; 95% CI: 0.41, 1.02). No data on outcomes such as quality of life or stroke recurrence are available. (Bath P M W, Bath S J, Asplund K. Pentoxifylline, propentofylline and pentifylline in acute ischaemic stroke. Cochrane Review [Updated 11 June 1996]. In: The Cochrane Library, Issue 4. Oxford: Update Software, 1998).

We observed three patients with minor CVA, TIA and ischemic stroke to demonstrate the efficacy of trental as a preventive agent for TIA/stroke. The first case was a 63-year-old male non-smoker who had previously undergone valve replacement surgery in 1987 and had received a coronary artery stent in 2002. The patient had been treated with coumudin since the first surgery. NR2 antibodies measured at base line were below the 2 ng/ml cutoff. (FIG. 1). Repeated blood assessments revealed a sudden increase of NR2 antibodies that showed a tendency toward further increases. At the same time, the patient had complaints on chronic fatigue and numbness in the 4^{th} and 5^{th} digits of his left arm lasting quite a long period of time. Oral trental taken in 200 mg twice a day was initiated for 30 days, beginning at the point designated by an arrow in FIG. 1, and reduced the level of NR2 antibodies by the end of treatment. Antibodies continued to decrease up to 6^{th} mo. after treatment was initiated and remained below the cut off level more than 1 year after therapy.

The second patient was a 66-year-old apparently healthy woman. Her blood assay for NR2 antibodies was performed initially on a voluntary basis as a healthy control. Slightly increased antibody levels above the 2.0 ng/ml cutoff were detected 1 mo before TIA onset (March 2002) that resulted in disorientation, and loss of consciousness for about 1 hour. DWI performed a week after TIA onset noted an asymmetry of the internal artery, neurological observation; EEG, EKG, and carotid ultrasound showed no abnormalities. However, NR2 antibodies detected within a week of onset were drastically increased (FIG. 2). Oral trental taken in 200 mg twice a day for 30 days reduced the level of NR2 antibodies to the control level of healthy individuals. Antibody monitoring for 1 year showed steady normal amounts that remains the same up to 2005. The patient used Plavix and Lipitor on daily basis as well.

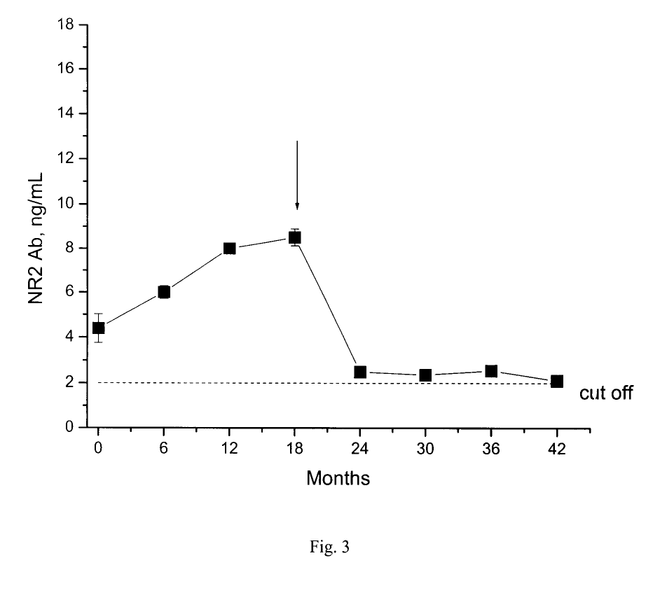

The third patient was an 84-year-old man, non-smoker who had suffered ischemic stroke on May 28-29, 2002 after angioplasty. He had receiving coumadin, eardizem, atenolol, hytrin, lasix, and guaifinex when he entered the study. NR2 antibodies measured at base line showed high antibody values above the cut off of 2 ng/ml (FIG. 3). Repeated blood tests revealed a trend of increasing NR2 antibodies that indicated a high risk of repetitive stroke. Oral Trental (200 mg×3 times a day) was initiated for 60 days and reduced the level of NR2 antibodies to the cut off Antibody levels remained below the cut off level up to 1 year after the therapy.

### Example 3

### NMDA Receptor Peptide and Antibodies for Assessing Pulmonary System in Neonates with Congenial Heart Diseases Requiring Cardiac Surgery

Thirteen infants undergoing cardiac surgery (study group) were prospectively studied. NR2/NR3 peptide and antibodies were detected in all patients at 2 time points; (1) baseline before surgery; (2) 24-hours postop. Data obtained were compared to NR2/NR3 peptide/antibodies in normal infants (n=4, control group). The normal infants were having elective surgery for hernia or congenital cataracts. The average age was 1.6±2.3 months in the study group compared to 2.8±1.7 months in the control group control group. Cardiac operations included: TOF repair (3); ASO with VSD repair (1); ASO (1); CAVC repair (1); TOF/PA complete repair (1); TAPVR repair (1); hypoplastic

TABLE 4
NR2/NR3 peptide/Ab levels at baseline and 24 hours
after cardiac surgery

| | | | | |
|---|---|---|---|---|
| Operation | Baseline | Baseline | 24 hr postop | 24 hr postop |
| | NR2/NR3 | NR2/NR3 Ab | NR2/NR3 | NR2/NR3 Ab |
| | (ng/ml) | (ng/ml) | (ng/ml) | (ng/ml) |

aortic arch repair (1); DCRV and VSD repair (1); coarctation repair and PAB (1); coarctation repair (1); Norwood (1). Baseline NR2/NR3 antibody levels were significantly higher in the study group compared to the control group (NR2/NR3 antibody p=0.002). NR2/NR3 peptide and antibodies levels at 24 hrs postop were statistically different compared to control group (NR2/NR3 peptide p=0.002; NR2/NR3 antibodies p=0.006). See Tables.

These data suggests that infants with congenital heart disease have elevated NR2/NR3 peptide and antibody at baseline compared to normal infants. This supports speculation that infants with congenital heart disease may have brain ischemia/injury prior to surgical intervention.

| | | | | |
|---|---|---|---|---|
| CAVC repair | 0.6 | 1.54 | 0.2 | 1.22 |
| PA/VSD repair | 0.2 | 1.8 | 0.4 | 1.8 |
| TAPVR repair | 0.2 | 1.11 | 1.1 | 1.22 |
| TOF repair | 1.2 | 1.66 | 0.9 | 1.57 |
| ASO/VSD repair | 1.3 | 1.66 | 1.2 | 1.62 |
| ASO | 8.9 | 1.5 | 2.2 | 1.53 |
| TOF repair | 0.2 | 1.46 | 0.6 | 1.52 |
| Aortic arch repair | 0.4 | 1.55 | 1.6 | 1.6 |
| DCRV/VSD repair | 1 | 1.2 | 0.8 | 0.8 |
| Coarctation | 0.4 | 0.6 | 0.2 | 0.5 |
| repair/PAB | | | | |
| Coarctation repair | 2.3 | 1 | 1.9 | 0.6 |
| Norwood | 0.3 | 0.7 | 1.3 | 1.9 |
| TOF repair | 0.26 | 0.48 | 0.1 | 0.30 |

Fallot;
ASO = arterial switch operation;
DCRV = double chamber right ventricle;
PAB = pulmonary artery band
CAVC = complete atrioventricular canal;
PA = pulmonary atresia;
VSD = ventricular septal defect;
TAPVR = total anomalous pulmonary venous return;
TOF = tetralogy of

TABLE 5
NR2/NR3 peptide/Ab at baseline and 24 hours
following cardiac surgery

| | | NR2/NR3 |
|---|---|---|
| | NR2/NR3 peptide ng/ml | antibody ng/ml |
| Control Group, N = 4 | 0.23 ± 0.13 | 0.73 ± 0.15 |
| Study Group | 1.33 ± 0.36 | 1.25 ± 0.44* |
| Baseline, N = 13 | | |
| Study Group | 0.96 ± 0.67^{#} | 1.24 ± 0.53^{#} |
| 24 hrs Postop, N = 13 | | |

*p < 0.05; control group vs baseline congenital heart disease
^{#}p < 0.05; control group vs 24 hour postop
2-tailed T-test

### Example 4

### Performance Characteristics of CIS-LA Antibody Test

Adult patients scheduled for CPB (cardio-pulmonary bypass) surgery were evaluated for recombinant NR2 antibody levels in serum before, and subsequently evaluated for neurological complications within 48 hours after the surgery. Table 6 presents the pre-op NR2 Ab at different cut offs depending on post-operative adverse event. The neurological adverse events group included patients with confusion, TIA and stroke (NIHSS of >9 scores). Patients who had no neurological event were assigned to the group of entitled “No Neuro Event”.

TABLE 6
Neurological Complications (TIA/Stroke) Pre-Op
or within 48 Hours vs. Pre-op NR2 Ab

| | | Neuro Event | No Neuro Event |
|---|---|---|---|
| Pre-Op NR2 Ab | | n/N (%) | n/N (%) |
| <1.5 | ng/mL | 7/213 (3.3%) | 206/213 (96.7%) |
| 1.5 to < 2.0 | ng/mL | 12/159 (7.6%) | 147/159 (92.5%) |
| ≧2.0 | ng/mL | 25/26 (96.2%) | 1/26 (3.9%) |

Table 7 shows detailed analyses of six different cut offs for NR2 Ab concentrations from 1.5 to 2.0 ng/ml. Although the event rate increases in the cut offs from 1.5 to 2.0 for both groups, it increases faster in the “Neuro Event” group. Therefore, the risk ratio increases significantly over the analyzed range with the best risk ratio corresponding to 2.0 ng/mL (ClinChem, 2003). 96.0% (24/25) of patients with NR2 Ab concentrations ≧2.0 ng/ml preoperatively had neurological complications within 48 hours post-CPB, vs. only 5.4% of patients with NR2 Ab concentrations <2.0 ng/ml, resulting in a 17.9-fold increase (95% CI, 11.6-27.6) in the predictive ability of a postoperative neurological adverse events.

TABLE 7
NR2 Ab results subdivided by different cut-offs

| | | | | | | Risk Ratio¹ |
|---|---|---|---|---|---|---|
| | | Neuro Event | | No Neuro Event | | (Lower 95% |
| Pre-Op NR2 Ab | | n/N (%) | | n/N (%) | | Bound)² |
| <1.5 | ng/mL | 8/214 | (3.7%) | 206/214 | (96.3%) | 5.2 |
| ≧1.5 | ng/mL | 36/184 | (19.6%) | 148/184 | (80.0%) | (2.8) |
| | | | | | | re 3 — |
| <1.6 | ng/mL | 10/288 | (3.5%) | 278/288 | (96.5%) | 8.9 |
| ≧1.6 | ng/mL | 34/110 | (30.9%) | 76/110 | (69.1%) | (5.1) |
| <1.7 | ng/mL | 12/319 | (3.8%) | 307/319 | (96.2%) | 10.8 |
| ≧1.7 | ng/mL | 32/79 | (40.5%) | 47/79 | (59.5%) | (6.4) |
| <1.8 | ng/mL | 17/351 | (4.8%) | 334/351 | (95.2%) | 11.9 |
| ≧1.8 | ng/mL | 27/47 | (57.5%) | 20/47 | (42.6%) | (7.6) |
| <1.9 | ng/mL | 19/364 | (5.2%) | 345/364 | (94.8%) | 14.1 |
| ≧1.9 | ng/mL | 25/34 | (73.5%) | 9/34 | (26.5%) | (9.4) |
| <2.0 | ng/mL | 20/373 | (5.4%) | 353/373 | (94.6%) | 17.9 |
| ≧2.0 | ng/mL | 24/25 | (96.0%) | 1/25 | (4.0%) | (12.4) |

¹Ratio of Event rate among patients with a positive pre-op NR2 Ab divided by event rate among patients with a negative pre-op NR2 Ab;
²Lower one-siced 95% confidence bound on the risk ratio.

Based on the obtained likelihood ratio of a neuro event of 17.9, a NR2 antibody concentration exceeding 2.0 ng/ml detected pre-operatively can predict neurological complications in 89% of patients after surgery. FIG. 4 presents three ROC curves, treating the pre-op tests as diagnostics to create the ROC curve. The area under the curve for pre-op N2 Ab indicates high predictive ability (AUC=0.814) for neurological adverse events before surgery. Preoperative NR2 Ab had high predictive value for TIA/stroke before CPB. The concentrations of NR2 Ab in serum samples from patients with no adverse events (neurocode 0) maintained under the cut off of 2.0 ng/ml at all time points of the study. In contrast, most patients with neurological adverse events (NIHSS scores>9) had increased NR2 Ab values above the cut off of 2.0 ng/ml pre-op, 24 hours and 48 hours after the procedure.

Throughout this application, various publications are referenced. The disclosures of these publications in their entireties are hereby incorporated by reference into this application in order to more fully describe the state of the art to which this invention pertains. It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1-15. (canceled)

16) An isolated peptide comprising SEQ ID NO:1, SEQ ID NO:2, SEQ ID NO:3 OR SEQ ID NO:4, or an analog thereof.

17) The peptide of claim 16 linked to a diagnostic substrate or indicator reagent.

18) The isolated peptide of claim 16 comprising SEQ ID NO:1.

19) The isolated peptide of claim 16 comprising SEQ ID NO:2.

20) The isolated peptide of claim 16 comprising SEQ ID NO:3.

21) The isolated peptide of claim 16 comprising SEQ ID NO:4.

22) An isolated antibody that is specific for a peptide comprising SEQ ID NO:1, SEQ ID NO:2, SEQ ID NO:3 OR SEQ ID NO:4, or an analog thereof.

23) The isolated antibody of claim 22 linked to a diagnostic substrate or indicator reagent.

24) The isolated antibody of claim 22 wherein said peptide has SEQ ID NO:1.

25) The isolated antibody of claim 22 wherein said peptide has SEQ ID NO:2.

26) The isolated antibody of claim 22 wherein said peptide has SEQ ID NO:3.

27) The isolated antibody of claim 22 wherein said peptide has SEQ ID NO:4.

28) An isolated nucleic acid that encodes for a peptide comprising SEQ ID NO:1, SEQ ID NO:2, SEQ ID NO:3 OR SEQ ID NO:4, or an analog thereof.

29-42. (canceled)
